(19) United States
(12) Patent Application Publication
(10) Pub. No.: US 2004/0266787 A1
(43) Pub. Date: Dec. 30, 2004

(54) Novel amorphous form of [2-[4-[(4-chlorophenyl)-phenyl methyl]-1-piperazinyl]ethoxy]acetic acid and process for the preparation thereof

(75) Inventors: Manne Satyanarayana Reddy, Hyderabad (IN); Srinivasan Thirumalai Rajan, Hyderabad (IN); Uppala Venkata Bhaskara Rao, Hyderabad (IN); Konda Srinivasa Reddy, Hyderabad (IN)

Correspondence Address: Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/809,193

(22) Filed: Mar. 25, 2004

(30) Foreign Application Priority Data
Mar. 25, 2003 (IN) 253/MAS/2003

(51) Int. Cl. A61K 31/495; C07D 241/04

(52) U.S. Cl. 514/255.04; 544/396

(57) ABSTRACT

A novel amorphous form of cetirizine and processes for making the amorphous form as well as compositions, pharmaceutical compositions, and methods utilizing the crystalline form are described.

### CROSS-REFERENCE TO RELATED APPLICATION

This application claims priority of Indian Patent Application No. 253/MAS/2003, filed Mar. 25, 2003, of which entire content is incorporated by reference.

### BACKGROUND OF THE INVENTION

Cetirizine is an orally active, long-acting histamine H₁ receptor antagonist. It belongs to the second generation of H₁ histamine receptor antagonists that are thought to offer some significant advantages over the first generation compounds. The advantages are believed to include less sedation, low anticholinergic activity, and longer acting duration with the resulting improves patient compliance. Cetirizine is used for the treatment of allergic syndromes, such as chronic and acute allergic rhinitis including seasonal and perennial allergic rhinits, allergic conjunctivitis, pruritus, urticaria, and the like.

### SUMMARY OF INVENTION

The invention relates to an amorphous form of cetirizine. Preferably, the amorphous form of cetirizine may have substantially the same X-ray diffraction pattern as shown in FIG. 1. Various embodiments and variants are provided.

The invention also relates to a composition that comprises cetirizine in a solid form, wherein at least 80% by weight of the solid cetirizine is an amorphous form of cetirizine.

The invention also relates to a process for preparation of an amorphous form of cetirizine.

The invention also relates to a pharmaceutical composition that comprises an amorphous form of cetirizine and one or more pharmaceutically acceptable carriers or diluents. The pharmaceutical composition may comprise one or more additional active ingredients in addition to cetirizine. Preferably, the pharmaceutical composition is in a solid dosage form for oral administration, such as a tablet.

The invention also relates to a method of preventing or treating allergic syndromes, comprising administering to a patient in need of such treatment an effective amount of an amorphous form of cetirizine.

### DESCRIPTION OF THE ACCOMPANYING DRAWINGS

FIG. 1 is an X-ray powder diffractogram of an amorphous form of cetirizine.

FIG. 2 is an infrared spectrum of an amorphous form of cetirizine.

FIG. 3 is a differential scanning calorimetry thermogram of an amorphous form of cetirizine.

### DETAILED DESCRIPTION OF THE INVENTION

Unless defined otherwise, all technical and scientific terms used herein have the same meaning as commonly understood by one of ordinary skill in the art, to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

For purposes of the present invention, the following terms are defined below.

A “compound” is a chemical substance that includes molecules of the same chemical structure.

“Pharmaceutically acceptable” means that which is useful in preparing a pharmaceutical composition that is generally non-toxic and is not biologically undesirable and includes that which is acceptable for veterinary use and/or human pharmaceutical use.

The term “composition” includes, but is not limited to, a powder, a suspension, an emulsion and/or mixtures thereof. The term composition is intended to encompass a product containing the specified ingredients in the specified amounts, as well as any product, which results, directly or indirectly, from combination of the specified ingredients in the specified amounts. A “composition” may contain a single compound or a mixture of compounds.

The term “pharmaceutical composition” is intended to encompass a product comprising the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

When referring to a chemical reaction, the terms “treating”, “contacting” and “reacting” are used interchangeably herein and refer to adding or mixing two or more reagents under appropriate conditions to produce the indicated and/or the desired product. It should be appreciated that the reaction, which produces the indicated and/or the desired product, may not necessarily result directly from the combination of two reagents, which were initially added, i.e., there may be one or more intermediates which are produced in the mixture which ultimately leads to the formation of the indicated and/or the desired product. Also, the term “isolating” is used to indicate separation of the compound being isolated regardless of the purity of the isolated compound from any unwanted substance which presents with the compound as a mixture. Thus, degree of the purity of the isolated or separated compound does not affect the status of “isolating”.

The term “substantially free of” in reference to a composition, as used herein, means that said substance cannot be detected in the composition by methods known to those skilled in the art at the time of the filing of this application.

The term “cetirizine,” which is interchangeably used with the term “cetirizine free species”, means a compound with the chemical name of [2-[4-[(4-chlorophenyl)-phenyl methyl]-1-piperazinyl]ethoxy]acetic acid having the structure,

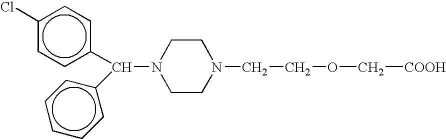

The preparation of cetirizine generally is known in the art. For example, the processes for the preparation of cetirizine and its dihysrochloride salt are disclosed U.S. Pat. No. 4,525,358, of which entire content is incorporated by reference herein. The disclosed process involves hydrolysis of the methyl ester of cetirizine using ethanolic potassium hydroxide to afford potassium salt of cetirizine. The solution containing the potassium salt is then acidified with hydrochloric acid. U.S. Pat. No. 6,255,487, incorporated by reference, discloses a process for the preparation of cetirizine dihydrochloride via condensation of (4-chloro phenyl) phenyl methyl chloride and potassium 2-(1-piperazinyl) ethoxyacetate in acetonitrile, followed by acidification in acetone medium with concentrated hydrochloric acid.

It is known that polymorphic forms of the same drug may have substantial differences in certain pharmaceutically important properties such as dissolution characteristics and bioavailability as well as stability of the drug. Furthermore, difference physical forms may have different particle size, hardness and glass transition temperature. Amorphous materials do not exhibit the three-dimensional long-range order found in crystalline materials but are structurally more similar to liquids where the arrangement of molecules is random. Amorphous solids are not crystalline and therefore do not give a definitive x-ray diffraction pattern (XRD), in addition they do not give rise to a melting point and tend to liquefy at some point beyond the glass transition point (Hancock and Zografi, (1997) J. Pharm. Sci., 86:1-12). Because amorphous solids do not have lattice energy, they dissolve in a solvent more rapidly and consequently provide a rapid bioavailability. Furthermore, amorphous forms of a drug may offer significant advantages over crystalline forms of the same drug in solid dosage form manufacture processes such as compressibility, economically or environmentally suitable solvents or process, or higher purity or yield of the desired product.

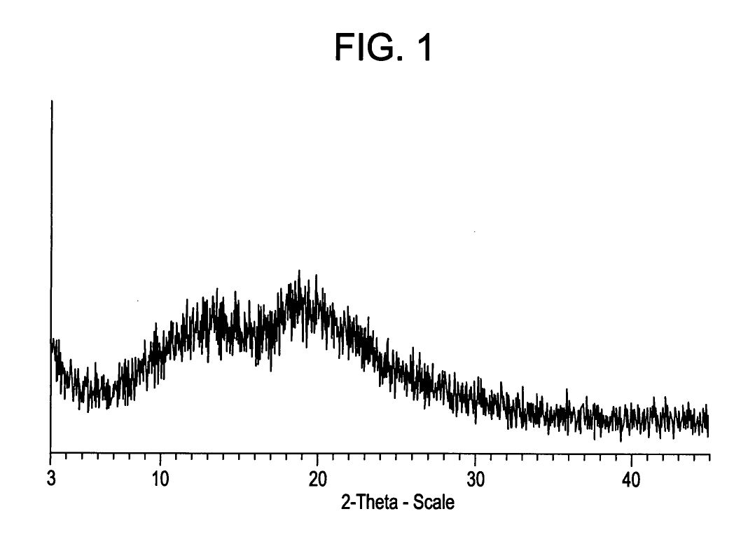

According to one aspect of the invention, there is provided cetirizine in an amorphous form. A sample of an XRD spectrum of cetirizine obtained by the inventors is shown in FIG. 1. As seen therefrom, the XRD pattern is highly characteristic of an amorphous solid. The X-ray diffractogram was measured on Bruker Axe, DS advance Power X-ray Diffractometer with Cu K alpha-1 Radiation source. A particular process for preparation of the amorphous form of cetirizine is also provided and includes: a) providing an aqueous solution of a water-soluble form of cetirizine; b) adjusting the pH of said aqueous solution to a range of from about 5 to about 5.5; c) contacting said aqueous solution with an extracting solvent selected from the group consisting of dichloromethane, chloroform, dichloroethane, ethyl acetate, methyl acetate and mixtures thereof; d) distilling off the solvent to form a solid residue; and e) isolating the solid residue to obtain the amorphous form of cetirizine.

The removal of the solvent from the cetirizine solution may be affected at an increased temperature, preferably at under reduced pressure. The solid residue obtained after the solvent removal may be isolated and dried using conventional methods. The advantages of the process include simplicity, eco-friendliness and suitability for commercial use. The aqueous solution of a water-soluble form of cetirizine can be obtained by dissolving a salt of cetirizine in water or alternatively by using [2-[4-[(4-chlorophenyl)-phenylmethyl]-1-piperazinyl]ethoxy]acetamide, which forms cetirizine in the presence of an alkaline base. When an amorphous form of cetirizine is obtained via [2-[4-[(4-chlorophenyl)-phenylmethyl]-1-piperazinyl]ethoxy]acetamide, the process may include a) reacting [2-[4-[(4-chlorophenyl)-phenylmethyl]-1-piperazinyl]ethoxy]acetamide with an alkaline base in water; b) adjusting the pH of the water to a range of from about 5 to about 5.5; c) extracting the cetirizine with a halogenated hydrocarbon solvents or acetate solvents; d) optionally drying the organic layer with a drying agent such as sodium sulphate, magnesium sulphate or molecular ceives; and e) distilling off the solvent, preferably under reduced pressure to afford the desired amorphous form of cetirizine. The amorphous form of cetirizine shown in FIG. 1 is produced by the described process.

The invention also relates to a composition of solid cetirizine wherein at least 80% of the total weight of cetirizine is in the amorphous form. In a preferred form of this composition, the solid cetirizine is suitable for use as a bulk active ingredient in formulating pharmaceutical products. The remainder of the solid cetirizine in the composition, i.e., 20% or less of the total weight of cetirizine, may be other forms of cetirizine, e.g. crystalline forms or polymorphs.

In an embodiment of the invention, the composition may include at least 95% of the amorphous form of cetirizine with respect to total weight of the solid cetirizine in the composition. In another embodiment of the invention, the composition may include at least 99% of the amorphous form of cetirizine with respect to total weight of the solid cetirizine in the composition. In yet another embodiment of the invention, the composition is substantially free of any forms of cetirizine other than its amorphous form. A crystalline form of cetirizine is disclosed in U.S. Pat. No. 4,525,358.

The preferred method of differentiating amorphous cetirizine from other crystalline and non-crystalline forms of cetirizine is X-ray powder diffraction (XPD). The XPD pattern of pure amorphous cetirizine, as illustrated in FIG. 1, can be seen to lack discernible acute peaks. Thus, amorphous cetirizine, according to the present invention, is characterized in providing an X-ray powder diffraction pattern containing one or more broad diffuse halos having very low counts (i.e. see FIG. 1) in contrast to the sharp diffraction peaks characteristic of crystalline materials. Of course it will be appreciated that a mixture comprising detectable amounts of both crystalline and amorphous cetirizine will exhibit both the characteristic sharp peaks and the diffuse halo(s) on XPD. This will be evident by an increase in the baseline and also a reduction in crystalline peak intensities.

X-ray diffraction also provides a convenient and practical means for quantitative determination of the relative amounts of crystalline and/or amorphous forms in a solid mixture. X-ray diffraction is adaptable to quantitative applications because the intensities of the diffraction peaks of a given compound in a mixture are proportional to the fraction of the corresponding powder in the mixture. The percent composition of amorphous or crystalline forms of cetirizine in an unknown composition can be determined. Preferably, the measurements are made on solid powder cetirizine. The X-ray powder diffraction patterns of an unknown composition can be compared to known quantitative standards containing pure crystalline forms of cetirizine to identify the percent ratio of a particular crystalline form. This is done by comparing the relative intensities of the peaks from the diffraction pattern of the unknown solid powder composition with a calibration curve derived from the X-ray diffraction patterns of pure known samples. The curve can be calibrated based on the X-ray powder diffraction pattern for the strongest peak from a pure sample of crystalline forms of cetirizine. The calibration curve may be created in a manner known to those of skill in the art. For example, five or more artificial mixtures of crystalline forms of cetirizine, at different amounts, may be prepared. In a non-limiting example, such mixtures may contain, 2%, 5%, 7%, 8%, and 10% of cetirizine for each crystalline form. Then, X-ray diffraction patterns are obtained for each artificial mixture using standard X-ray diffraction techniques. Slight variations in peak positions, if any, may be accounted for by adjusting the location of the peak to be measured. The intensities of the selected characteristic peak(s) for each of the artificial mixtures are then plotted against the known weight percentages of the crystalline form. The resulting plot is a calibration curve that allows determination of the amount of the crystalline forms of cetirizine in an unknown sample. For the unknown mixture of crystalline and amorphous forms of cetirizine, the intensities of the selected characteristic peak(s) in the mixture, relative to an intensity of this peak in a calibration mixture, may be used to determine the percentage of the given crystalline form in the composition, with the remainder determined to be the amorphous material.

In addition to X-ray powder diffraction, amorphous cetirizine, or the presence of some amorphous cetirizine, can be distinguished from crystalline cetirizine, using Raman spectroscopy, solution calorimetry, differential scanning calorimetry, solid state nuclear magnetic resonance spectra (ssNMR) or infra-red spectroscopy. Each of these techniques is well established in the art. Amorphous cetirizine can also be identified based on the morphology of the particles seen under an electron microscope. Furthermore, amorphous cetirizine is likely to be much more soluble than crystalline cetirizine because the former is lack of lattice energy, providing another means of discriminating between the crystalline and amorphous cetirizine forms, or detecting an amount of amorphous form within a cetirizine preparation. As noted above, the preferred method of differentiating amorphous cetirizine from other crystalline and non-crystalline forms of cetirizine is X-ray powder diffraction (XPD).

Another method of distinguishing physical forms, such as crystalline and amorphous cetirizine, is ¹³C solid state NMR spectra (ssNMR) acquired with cross polarization, magic angle spinning and high power proton decoupling. The isotropic chemical shifts (peak positions) measured in solid state NMR spectra are not only a function of the molecule's atomic connectivity, but also of molecular conformation and inter- and intra-molecular interactions. Thus different peak positions may be observed for different physical forms. For amorphous materials, the dispersion of environments often causes substantially broadened spectra.

It will be appreciated that because of the enhanced solubility property of amorphous cetirizine, mixtures comprising substantially crystalline or other solid forms of cetirizine with amorphous cetirizine will, depending on the amount of amorphous product present, may also possess varying degrees of increased solubility. Such mixtures comprising amorphous cetirizine can be prepared, for example, by mixing amorphous cetirizine prepared according to the present invention with other solid forms of cetirizine, such as crystalline form, prepared according to prior art methods. A mixture might also be prepared if the manufacturing process is incomplete, or incorporates steps that allow or cause amorphous product to be formed. Examples of other solid forms of cetirizine include, but are not limited to, crystalline cetirizine, and other polymorphs. A detectable amount of amorphous cetirizine is an amount that can be detected using conventional techniques, such as FT-IR, Raman spectroscopy, XPD, TMA, DSC and the like.

As noted above, numerous techniques can be employed to detect a particular form of a compound within a mixture. The limits of detection of a particular form in admixture with another form, i.e. crystalline in amorphous or vice versa, are as follows: by XPD it is reported to be approximately 5% according to Hancock and Zografi (J. Pharm. Sci., 86:1-12, 1997) and approximately 2.0% according to Surana and Suryanarayanan (Powder Diffraction, 15:2-6, 2000). The limit of detection by solution calorimetry is reported to be approximately 1% according Hogan and Buckton (International Journal of Pharmaceutics, 207:57-64, 2000). The limit of detection by solid state NMR is reported to be approximately 5-10% according to Saindonet al., (Pharmaceutical Research, 10:197-203, 1993). The limit of detection by near infrared spectroscopy is reported to be approximately 2-5% according to Blanco and Villar (Analyst, 125:2311-2314, 2000). The limit of detection by Modulated Differential Scanning Calorimetry (MDSC) is reported to be approximately 6% according to Saklatvala et al., (International Journal of Pharmaceutics, 192: 55-62, 1999). The limit of detection by FTRaman spectroscopy is reported to be approximately 2% according to Taylor and Zografi (Pharm. Res. 15:755-761, 1998).

In another embodiment, the invention provides pharmaceutical compositions comprising the amorphous form of cetirizine, which can be formulated with a one or more pharmaceutically acceptable carriers, also known as excipients, which ordinarily lack pharmaceutical activity, but have various useful properties which may, for example, enhance the stability, sterility, bioavailability, and ease of formulation of a pharmaceutical composition. These carriers are pharmaceutically acceptable, meaning that they are not harmful to humans or animals when taken appropriately and are compatible with the other ingredients in a given formulation. The carriers may be solid, semi-solid, or liquid, and may be formulated with the compound in bulk. The resulting mixture may be manufactured in the form of a unit-dose formulation (i.e., a physically discrete unit containing a specific amount of active ingredient) such as a tablet or capsule. The pharmaceutical compositions may include, in addition to a compound of this invention, one or more active pharmaceutical compounds.

The pharmaceutical compositions may include, in addition to a compound of this invention, one or more active pharmaceutical compounds. For example, U.S. Patent Publication No. 2002/0012700, incorporated by reference, discloses a combination dosage form comprising cetirizine and pseudoephedrine. Similarly, U.S. Patent Publication No. 2002/0099058, incorporated by reference, discloses pharmaceutical compositions containing cetirizine and a leukotriene inhibitor and its pharmaceutically acceptable salts such as zileuton. Also U.S. Pat. No. 4,829,064, incorporated by reference, discloses compositions useful for treating cold symptoms comprising cetirizine and an analgesic. Thus, the amorphous form of the present invention may also be combined with pseudoephedrine, a leukotriene inhibitor or an analgesic to utilize the advantages of the present invention.

Generally, the pharmaceutical compositions of the invention may be prepared by uniformly admixing the active ingredient with liquid or solid carriers and then shaping the product into the desired form. The pharmaceutical compositions may be in the form of suspensions, solutions, elixirs, aerosols, or solid dosage forms. Because of their ease of administration, tablets and capsules represent the most advantageous oral dosage unit form, in which case solid pharmaceutical carriers are employed.

A preferred oral solid preparation is a tablet. A tablet may be prepared by direct compression, wet granulation, or molding, of the active ingredient(s) with a carrier and other excipients in a manner known to those skilled in the art. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made on a suitable machine. A mixture of the powdered compound moistened with an inert liquid diluent is suitable in the case of oral solid dosage forms (e.g., powders, capsules, and tablets). If desired, tablets may be coated by standard techniques. The compounds of this invention may be formulated into typical disintegrating tablets, or into controlled or extended release dosage forms.

The pharmaceutical compositions of the invention are contemplated in various formulations suitable for various modes of administration, including but not limited to inhalation, oral, rectal, parenteral (including subcutaneous, intradermal, intramuscular, intravenous), implantable, intravaginal and transdermal administration. The most suitable route of administration in any given case depends on the duration of the subject's condition, the length of treatment desired, the nature and severity of the condition being treated, and the particular formulation that is being used. The formulations may be in bulk or in unit dosage form.

The amount of active ingredient included in a unit dosage form depends on the type of formulation that is formulated. A pharmaceutical composition of the invention will generally include about 0.1% by weight to about 99% by weight of active ingredient, preferably about 1% by weight to 50% by weight for oral administration and about 0.2% by weight to about 20% by weight for parenteral administration.

Formulations suitable for oral administration include capsules (hard and soft), cachets, lozenges, syrups, suppositories, and tablets, each containing a pre-determined amount of the active compound; as a powder or granules; as a solution or a suspension in an aqueous or non-aqueous liquid; or as an oil-in-water or water-in-oil emulsion. Such formulations may be prepared by any suitable method of pharmacy that includes the step of bringing into association the active compound and a suitable carrier or carriers. The amount of active ingredient per unit dosage of solid formulations may be as described in prior art for preparations of cetirizine. For liquid oral formulations, a preferable amount is from about 2% by weight to about 20% by weight. Suitable carriers include but are not limited to fillers, binders, lubricants, inert diluents, surface active/dispersing agents, flavorants, antioxidants, bulking and granulating agents, adsorbants, preservatives, emulsifiers, suspending and wetting agents, glidants, disintegrants, buffers and pH-adjusting agents, and colorants. Examples of carriers include celluloses, modified celluloses, cyclodextrins, starches, oils, polyols, sugar alcohols and sugars, and others. For liquid formulations sugar, sugar alcohols, ethanol, water, glycerol, and polyalkylene glycols are particularly suitable, and may also be used in solid formulations. Cyclodextrins may be particularly useful for increasing bioavailability. Formulations for oral administration may optionally include enteric coatings known in the art to prevent degradation of the formulation in the stomach and provide release of the drug in the small intestine. One example of pharmaceutical tablet of the amorphous cetirizine may include, as inactive ingredients, hypromellose 2910, lactose monohydrate, magnesium stearate, microcrystalline cellulose, polyethylene glycol 3000, sodium starch glycolate, titanium dioxide, triacetin and 1 or more of synthetic red and yellow iron oxides and talc.

Formulations suitable for buccal or sub-lingual administration include lozenges comprising the active compound in a flavored base, usually sucrose and acacia or tragacanth, although other agents are also suitable, and pastilles comprising the compound in an inert base such as gelatin and glycerin or sucrose and acacia.

Formulations suitable for rectal administration are preferably presented as unit dose suppositories. These may be prepared by admixing the active compound with one or more conventional solid carriers, e.g., cocoa butter, and then shaping the resulting mixture.

The effective amount (i.e., dosage) of active compound for treatment will vary depending on the route of administration, the condition being treated, its severity, and duration, and the state and age of the subject. A skilled physician will monitor the progress of the subject and will adjust the dosage accordingly, depending on whether the goal is to eliminate, alleviate, or prevent a given condition. Generally, the dosage should be considered in proportion to the subject's weight. The daily dose of particular formulations of active compound may be divided among one or several unit dose administrations. For example therapeutic administration about fifteen to thirty minutes before main meals is preferable (i.e. three times daily), although administration of the active compounds may be carried out prophylactically, and may be maintained for prolonged periods of time. One skilled in the art will take such factors into account when determining dosage. Unit dosage of active ingredient may range from about 0.1 mg to about 2 g, preferably from about 1 mg to about 1 g, more preferably from about 1 mg to about 10 mg, even more preferably from about 2 mg to about 8 mg.

Since the amorphous form of cetirizine of the present invention does not have the lattice energy, it may easily form a dispersion in water. Thus, the amorphous form of cetirizine may also serve as a effective starting material to make other salt forms of cetirizine such as the dihydrochloride salt. Another aspect of the invention also provides a process of making cetirizine dihydrochloride, which may include: a) providing a solid powder which is a cetirizine free species in an amorphous form; b) contacting said solid powder with a liquid phase containing water; and c) adding two or more quivalents of hydrochloric acid to said liquid phase so that said cetirizine free species is converted to said cetirizine dihydrochloride.

In another aspect, the invention also provides methods of preventing or treating a treatment of allergic syndromes, such as chronic and acute allergic rhinitis including seasonal and perennial allergic rhinits, allergic conjunctivitis, pruritus, urticaria, and the like.

The effective amount (i.e., dosage) of active compound for treatment will vary depending on the route of administration, the condition being treated, its severity, and duration, and the state and age of the subject. A skilled physician will monitor the progress of the subject and will adjust the dosage accordingly, depending on whether the goal is to eliminate, alleviate, or prevent a given condition. Generally, the dosage should be considered in proportion to the subject's weight. The daily dose of particular formulations of active compound may be divided among one or several unit dose administrations. For example therapeutic administration about fifteen to thirty minutes before main meals is preferable (i.e. three times daily), although administration of the active compounds may be carried out prophylactically, and may be maintained for prolonged periods of time. One skilled in the art will take such factors into account when determining dosage. Unit dosage of active ingredient may range preferably from about 1 mg to about 100 mg, more preferably from about 10 mg to about 50 mg.

The invention is further described by reference to the following examples which set forth in detail the preparation of compounds and compositions of the present invention, as well as their utility. It will be apparent to those skilled in the art, that many modifications, both to materials, and methods, may be practiced without departing from the purpose and interest of this invention. The examples that follow are not intended to limit the scope of the invention as described hereinabove or as claimed below.

### EXAMPLE 1

### Preparation of Novel Amorphous Form of Cetirizine

2-[4-[(4-Chlorophenyl-phenyl methyl)-1-piperazinyl]ethoxy]acetamide (50 grams) was charged into a solution of sodium hydroxide (12.9 grams) and water (200 ml). The reaction mass was heated to reflux and maintained at reflux for 12 hours. The reaction mass was cooled to 50-60° C. and water (300 ml) was added in order to further cool the reaction solution to 20-25° C., of which the pH was subsequently adjusted to 9.5 to 9.8 with hydrochloric acid. The aqueous solution was washed with ethyl acetate (200 ml), and pH of the aqueous solution further adjusted to 7.0-7.5 with hydrochloric acid. Water (100 ml) was distilled off from the aqueous solution under reduced pressure at 60-80° C. Fresh water (100 ml) was charged and was again distilled off (50 ml) under reduced pressure at 60-80° C. Fresh water (50 ml) was added to the residue, and the pH of the resulting solution was adjusted to 5-5.5 with hydrochloric acid at 25-35° C. Extractions were given to the aqueous solution with dichloromethane (2×150 ml), followed by washing the combined organic solution with water (2×150 ml) and 10% sodium chloride solution. The organic layer was separated and treated with charcoal at reflux temperature for 15-20 minutes. The reaction mass was filtered through hyflow bed and washed with dichloromethane (50 ml). The organic solution was dried under sodium sulphate, and the organic solvent was distilled off under reduced pressure to get the amorphous form of cetirizine (32.1 grams). Melting point 45-48° C.

### EXAMPLE 2

2-[4-[(4-Chlorophenyl) phenyl methyl]-1-piperizinyl]ethanol (50.0 grams) was charged into a mixture of dimethyl formamide (150.0 ml), potassium hydroxide (25.4 gram) sodium monochloroacetate (26.4 gram) and maintained at 25-35° C. for 10-12 hours. And water (500 ml) was added to the reaction mass followed by washing the aqueous layer with toluene (4×100 ml). Then aqueous layer pH was adjusted to 5-5.5 with hydrochloric acid at 25-35° C. Extractions were given to the aqueous solution with dichloromethane (2×150 ml) followed by washing the combined organic solution with water (2×150 ml) and 10% sodium chloride solution. The organic layer was separated and treated with charcoal at reflux temperature for 15-20 minutes. The reaction mass was filtered through hyflow bed and washed the bed with dichloromethane (50 ml). The organic solution was dried under sodium sulphate, and the organic solvent was distilled off under reduced pressure to get the amorphous form of cetirizine (31.1 grams). Melting point: 45-48° C.

### EXAMPLE 3

### Preparation of Cetirizine Dihydrochloride from Solid Amorphous Cetirizine Free Species

A solid powder of cetirizine free species (10.0 grams) is dissolved in ethyl acetate (100 ml) at a temperature of 25-35° C. and stirred for 10-15 min. Isopropanolic hydrochloric acid (20 ml) is added till the pH of reaction mass becomes 2.0. The reaction mass is stirred for 1-2 hours to separate the solid. The separated solid is filtered, washed with ethyl acetate (20 ml), followed by hexane (10 ml) and on subsequent drying at a temperature of 80-100° C. to a constant weight provides solid of cetirizine dihydrochloride.

### EXAMPLE 4

### Soluble Granules Containing an Amorphous Cetirizine

Soluble granules containing an amorphous cetirizine may have the following content:

| Ingredient | Content (mg) |
|---|---|
| Amorphous cetirizine | 10 |
| Calcium carbonate | 800 |
| Citric acid | 900 |
| Avicel | 40 |
| Mannitol | 625 |
| Maltodextrin | 15 |
| Aspartame | 3 |
| Aroma | 20 |

### EXAMPLE 5

### Dispersible Tablet Containing an Amorphous Cetirizine

Dispersible tablet containing an amorphous cetirizine may have the following content:

| Ingredient | Content (mg) |
|---|---|
| Amorphous cetirizine | 10 |
| Calcium carbonate | 500 |
| Polyvinylpyrrolidone | 17 |
| Avicel | 15 |
| Mannitol | 400 |
| Maltodextrin | 15 |
| Aspartame | 3 |
| Aroma | 20 |

We claim:

1. A compound which is a cetirizine free species in an amorphous form.

2. The compound of claim 1 having substantially the same X-ray diffraction pattern as shown in FIG. 1.

3. The compound of claim 1 having an infrared absorption spectrum comprising absorption bands at about 3414 cm⁻¹, about 2828 cm⁻¹, about 2459 cm⁻¹, about 1159 cm⁻¹, and about 1488 cm⁻¹.

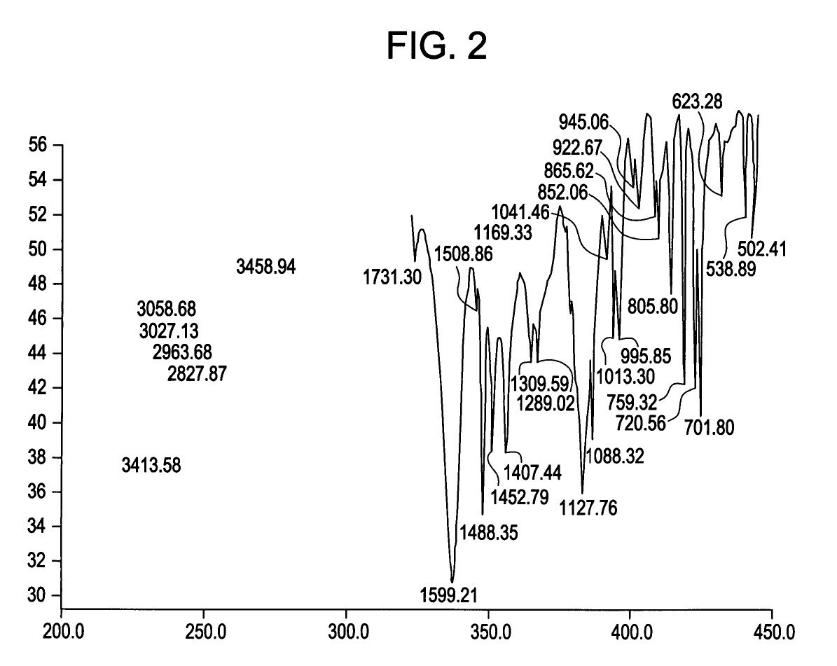

4. The compound of claim 1 having substantially the same infrared spectrum as shown in FIG. 2.

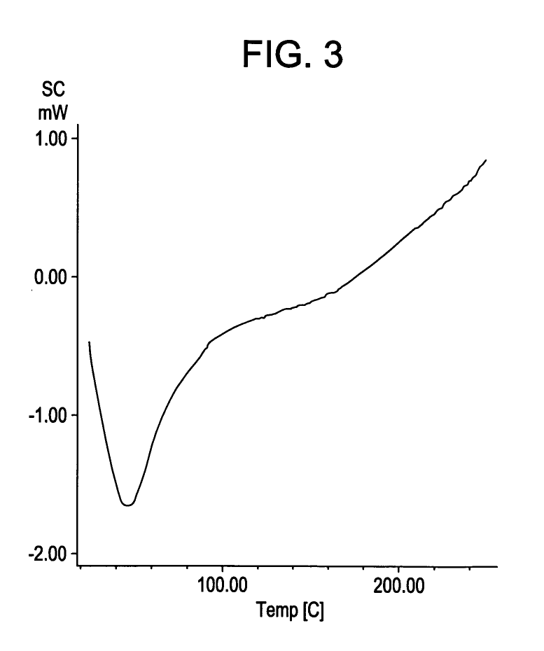

5. The compound of claim 1 having substantially the same differential scanning calorimetry thermogram as shown in FIG. 3.

6. A composition comprising cetirizine free species as a solid, wherein at least 80% by weight of said solid cetirizine is in an amorphous form.

7. The composition of claim 6, wherein at least 95% of said solid cetirizine is in said amorphous form.

8. The composition of claim 6, wherein at least 99% of said solid cetirizine is in said amorphous form.

9. The composition of claim 6, which is substantially free of crystalline forms of cetirizine free species.

10. A process for making an amorphous form of cetirizine free species, said process comprising:
a. providing an aqueous solution of a water-soluble form of cetirizine;
b. adjusting the pH of said aqueous solution to a range of from about 5 to about 5.5;
c. contacting said aqueous solution with an extracting solvent selected from the group consisting of dichloromethane, chloroform, dichloroethane, ethyl acetate, methyl acetate and mixtures thereof;
d. distilling off said solvent to form a solid residue; and
e. isolating said solid residue to obtain said amorphous form of cetirizine free species.

11. The process of claim 10, wherein said extracting solvent is dichloromethane.

12. A compound which is the amorphous form of cetirizine free species produced by the process of claim 10.

13. A compound which is the amorphous form of cetirizine free species produced by the process of claim 11.

14. A pharmaceutical composition comprising an amorphous form of cetirizine free species and one or more pharmaceutically acceptable carriers.

15. The pharmaceutical composition of claim 14, further comprising at least one additional active ingredient.

16. The pharmaceutical composition of claim 15, wherein said additional active ingredient is pseudoephedrine.

17. The pharmaceutical composition of claim 15, wherein said additional active ingredient is a leukotriene inhibitor.

18. The pharmaceutical composition of claim 15, wherein said additional active ingredient is an analgesic.

19. A method of treating allergic syndromes, which comprises administering a mammal in need thereof an effective amount of the compound of claim 1.

20. The method of claim 20, wherein said mammal is a human.

21. A process of making cetirizine dihydrochloride which comprises:
a) providing a solid powder which is a cetirizine free species in an amorphous form;
b) contacting said solid powder with a liquid phase containing water; and
c) adding two or more equivalents of hydrochloric acid to said liquid phase so that said cetirizine free species is converted to said cetirizine dihydrochloride.

22. The process of claim 21, further comprising dissolving said solid powder in an organic solvent prior to said contacting step.

23. The process of claim 22, wherein said organic solvent is selected from the group consisting of dichloromethane, chloroform, dichloroethane, ethyl acetate, methyl acetate and mixtures thereof.

24. The process of claim 22, wherein said hydrochloric acid is added in an alcoholic solution.
